(19) United States
(12) Patent Application Publication
(10) Pub. No.: US 2012/0143208 A1
(43) Pub. Date: Jun. 7, 2012

(54) Post-operative circumcision device

(76) Inventors: Noe Eduardo Perez, Laguna Vista, TX (US); Diane Perez, Laguna Vista, TX (US)

(21) Appl. No.: 12/928,094

(22) Filed: Dec. 3, 2010

(51) Int. Cl. A61B 17/326

(52) U.S. Cl. 606/118

(57) ABSTRACT

A unique post-operative circumcision device (POCD) is disclosed which reduces the possibility of discomfort or injury to the circumcised penis from outside external threats to the circumcised penis with such POCD being applied to encompass the circumcised penis of the patient. The POCD is to be applied immediately after circumcision post-operatively at the health care facility. The POCD in its preferred form is composed of a semi-pliable material suitable for human use in the shape of a cylindrical cup and placed to encompass the circumcised penis. The POCD is to be applied post-operatively before the patient applies clothing to reduce the possibility of externalities injuring or discomforting the circumcised penis. A disposable POCD is to be replaced four times daily or after each bath for up to five days after the date of circumcision or as suggested by the physician or surgeon. A non-disposable POCD can be used and must be disinfected four times daily or after each bath.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Our five year old son was diagnosed with phimosis on Sep. 12, 2010 which led to his circumcision in October of 2010. Post-operatively, we were sent home with instructions to bathe our son four times daily and to apply an antibiotic ointment on his circumcised penis after bathing. The problem was evident at the hospital. He was in a lot of pain and discomfort when his underwear or t-shirt touched or brushed against his circumcised penis. The only suggestions we were given at the hospital to alleviate discomfort was to allow him to walk around with no clothing or with just a t-shirt. The lack of mobility and embarrassment to our five year old son resulted in us coming up with a solution to this problem with the solution being a post-operative circumcision device composed of semi-pliable material suitable for human use.

The post-operative circumcision device was proven successful without the optional strap for our son. Additionally, the post-operative circumcision device reduced our son's discomfort and embarrassment by allowing him to wear his undergarment post-operatively. It is worth noting that our son successfully used the post-operative circumcision device we made for him out of a Styrofoam coffee cup. Our invention is not confined to the use of Styrofoam material in that other durable materials are available and could be used to accomplish the primary objective of our invention which is to reduce the possibility of post-operative injury or discomfort to the circumcised penis. Furthermore, we successfully constructed a post-operative circumcision device with an adjustable strap using elastic and Velcro. The use and description of the materials used in our invention does not limit the availability and use of other materials that can be used to construct our post-operative circumcision device with or without an optional strap in a manner that will accomplish the aforementioned primary objective of our invention.

For millennia, circumcisions have been performed for hygienic and/or religious reasons. Although the focus of circumcisions has been on how to circumcise the penis, attention has not been placed on reducing patient discomfort after circumcision using devices developed for that purpose.

Although the use of medicine is necessary and indispensable, medicine does not prevent clothing or other externalities from injuring the circumcised penis or from causing discomfort to the circumcised penis. Because the glans of the penis is sensitive after circumcision, patient discomfort can be reduced by use of a post-operative circumcision device that encompasses the circumcised penis for as much time as is needed while the circumcised penis heals.

Although circumcision is preferred for infant boys or young boys, it is also done on older males. Regardless of age, the discomfort or pain of circumcision can take time to dissipate during the healing process. The post-operative circumcision device can provide psychological benefits as the patient's confidence is increased because the patient is reassured that such a device will reduce the possibility of injury or discomfort to the circumcised penis. The use of the post-operative circumcision device by our five year old son proved to reduce discomfort and injury to his circumcised penis thereby allowing him to have greater mobility than would have otherwise been possible.

With less discomfort, circumcised patients may be able to attend school or work sooner as was the case with our son. It would be of value not only to the patient but to schools, businesses, and the medical profession to have a post-operative circumcision device that reduces the possibility of discomfort or injury to the circumcised penis. Thus, the primary objective of the present invention is to have a post-operative circumcision device that reduces the possibility of post-operative injury or discomfort to the circumcised penis.

### BRIEF SUMMARY OF THE INVENTION

Accordingly, a post-operative circumcision device is provided which is comprised of a cylindrical cup of semi-pliable material suitable for human use that has a sufficient opening on both ends of the post-operative circumcision device such that the device can be placed to encompass the circumcised penis. We prefer the post-operative circumcision device encompass the circumcised penis as soon as practicable after the circumcision to protect the circumcised penis.

We also prefer that the patient be provided with post-operative circumcision devices after the circumcision and that the patient or patient's legal guardians or parents be educated by medical staff at the health care facility how to place and use the post-operative circumcision device.

If the post-operative circumcision device is non-disposable, we prefer the patient or patient's legal guardians or parents be educated by medical staff at the health care facility on how to use and disinfect the device.

Refer to the figures of the drawing with the details attached hereto that detail the structure of the post-operative circumcision device and its placement encompassing the circumcised penis.

Once the post-operative circumcision device is placed encompassing the circumcised penis, the clothing of the patient is to be put over the post-operative circumcision device.

We recommend the disposable or non-disposable post-operative circumcision device be used for a period of at least five days after the date of circumcision or longer if needed by the patient or as suggested by the physician or surgeon. We recommend using a new disposable post-operative circumcision device four times daily or after each bath or as suggested by the physician or surgeon. We recommend that if a non-disposable post-operative circumcision device is used that it be disinfected four times daily or after each bath.

### BRIEF DESCRIPTION OF THE SEVERAL VIEWS OF THE DRAWING

The invention will be described by reference to FIGS. 1-6 of the drawing.

FIG. 1 shows a view of the post-operative circumcision device 2 in an upright position with attached adjustable straps 1 and 3. Strap attachment 4 is held securely in place.

FIG. 2 shows a view of the post-operative circumcision device 2 the way it will encompass the entire circumcised penis thereby reducing the possibility of contact with externalities that could cause discomfort or injury to the circumcised penis. This view shows the post-operative circumcision device 2 without the adjustable straps 1 and 3 which are optional.

FIG. 3 shows the post-operative circumcision device 2 in place against the patient's skin encompassing the patient's circumcised penis in a protecting manner with the adjustable straps 1 and 3 in place.

FIG. 4 shows the adjustable strap 1 and 3 with strap attachment 4.

FIG. 5 shows another view of the post-operative circumcision device 2 in place underneath the patient's undergarment or diaper thereby protecting the circumcised penis from coming into contact with clothing or diaper so as to reduce discomfort. The post-operative circumcision device 2 is held in place using the adjustable strap 1 showing.

FIG. 6 shows the same view as FIG. 5 of the post-operative circumcision device 2 in place underneath the patient's undergarment or diaper thereby protecting the circumcised penis from coming into contact with clothing or diaper so as to reduce discomfort. FIG. 6 does not include the adjustable strap 1 and 3, with the adjustable strap land 3 being optional to the post-operative circumcision device 2.

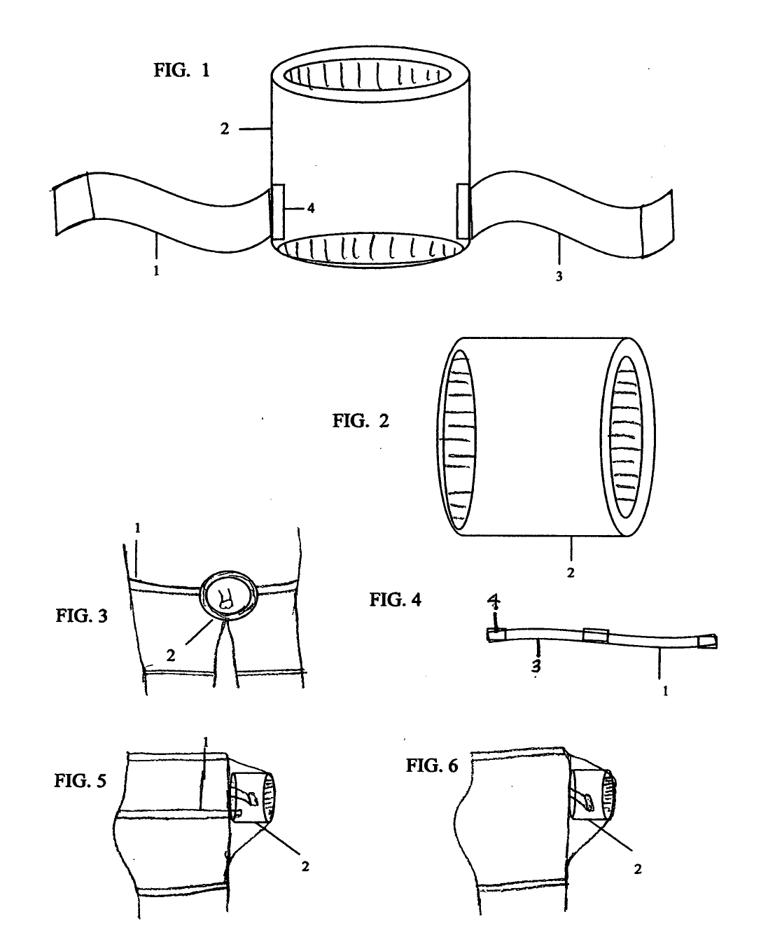

### DETAILED DESCRIPTION OF THE INVENTION

Before explaining the present invention in detail, it should be noted the embodiment of the invention is not confined to the regimental illustrations or drawings presented in this invention and that the present invention can be manifested in various forms and should be used as the primary objective intended which is to encompass the circumcised penis to reduce the possibility of post-operative discomfort or injury to the circumcised penis. Stringent adherence to the procedures for disinfecting the non-disposable post-operative circumcision device and for replacing disposable post-operative circumcision devices after each bath is recommended.

The post-operative circumcision device is constructed of semi-pliable material suitable for human use. In FIGS. 3, 5, and 6, the post-operative circumcision device is shown in its intended use. FIGS. 3 and 5 illustrate the post-operative circumcision device with the optional adjustable strap in place to hold the post-operative circumcision device more securely if desired by the patient. The adjustable strap is shown in FIG. 4. In FIG. 6, the post-operative circumcision device is being held in place by the patient's undergarment.

The post-operative circumcision device is constructed of a semi-pliable cylindrical cup with openings on both ends of the cup. The post-operative circumcision device used in our invention is not limited to a specific size but should be large enough to encompass an entire circumcised penis of a patient.

We claim as our invention:

1. A post-operative circumcision device which is comprised of semi-pliable material suitable for human use that is in the shape of a cylindrical cup with a circular opening on both ends with an optional adjustable strap to keep the device in place that is used to cover the circumcised penis to reduce the possibility of injury and discomfort to the circumcised penis from externalities.

2. According to claim 1, the post-operative circumcision device is to be placed over the circumcised penis for the purpose of protecting the circumcised penis immediately after circumcision at the health care facility where the circumcision occurred.

3. According to claim 1, the post-operative circumcision device is to be disposed of at least four times daily or after each bath unless the post-operative circumcision device is non-disposable, in which case, the post-operative circumcision device is to be disinfected four times daily or after each bath.

4. According to claim 1, the post-operative circumcision device is to be of the appropriate size to reduce the possibility of externalities injuring the circumcised penis or causing discomfort to the circumcised penis.
